(19) United States
(12) Patent Application Publication
(10) Pub. No.: US 2025/0120839 A1
(43) Pub. Date: Apr. 17, 2025

(54) UPPER ARM SPLINT

(71) Applicant: Safe Splints, LLC, Winter Park, FL (US)

(72) Inventor: Jeffrey A. Deren, Winter Park, FL (US)

(73) Assignee: Safe Splints, LLC, Winter Park, FL (US)

(21) Appl. No.: 18/912,904

(22) Filed: Oct. 11, 2024

Related U.S. Application Data
(60) Provisional application No. 63/543,633, filed on Oct. 11, 2023.

(51) Int. Cl. A61F 5/058

(52) U.S. Cl. CPC A61F 5/05816; A61F 5/05858

(57) ABSTRACT

Upper arm splint. An upper arm splint referenced herein includes a rigid outer member configured to fit upon an outside of an upper arm and shoulder, a rigid inner member configured to fit upon an inside of the upper arm, a plurality of outer stabilization bladders coupled to the rigid outer member and capable of independent inflation, a plurality of inner stabilization bladders coupled to the rigid inner member and capable of independent inflation, and a plurality of bands or straps connecting the rigid outer member to the rigid inner member.

### PRIORITY

The present application is related to, and claims the priority benefit of, U.S. provisional patent application Ser. No. 63/543,633, filed Oct. 11, 2023, the contents of which are incorporated herein directly and by reference in their entirety.

### BRIEF SUMMARY

The present disclosure includes disclosure of an upper arm splint device, comprising a rigid outer member configured to fit upon an outside of an upper arm and shoulder, a rigid inner member configured to fit upon an inside of the upper arm, a plurality of outer stabilization bladders coupled to the rigid outer member and capable of independent inflation, a plurality of inner stabilization bladders coupled to the rigid inner member and capable of independent inflation, and a plurality of bands or straps connecting the rigid outer member to the rigid inner member.

The present disclosure includes disclosure of an upper arm splint device, wherein the rigid outer member is capable of inflation, and wherein the rigid outer member becomes rigid upon inflation.

The present disclosure includes disclosure of an upper arm splint device, wherein the rigid inner member is capable of inflation, and wherein the rigid inner member becomes rigid upon inflation.

The present disclosure includes disclosure of an upper arm splint device, wherein the rigid outer member and/or the rigid inner member comprises plastic.

The present disclosure includes disclosure of an upper arm splint device, wherein the rigid outer member and/or the rigid inner member comprises metal.

The present disclosure includes disclosure of an upper arm splint device, wherein the plurality of bands or straps are coupled to the rigid outer member and are configured for subsequent connection to the rigid inner member.

The present disclosure includes disclosure of an upper arm splint device, wherein the plurality of bands or straps are coupled to the rigid inner member and are configured for subsequent connection to the rigid outer member.

The present disclosure includes disclosure of an upper arm splint device, configured to fit around an upper arm of a wearer having a broken humerus bone.

The present disclosure includes disclosure of an upper arm splint device, wherein inflation of one or more bladders of the plurality of outer stabilization bladders and/or the plurality of inner stabilization bladders causes the broken humerus bone to properly align to facilitate proper healing.

The present disclosure includes disclosure of an upper arm splint device, wherein inflation of two bladders of the plurality of outer stabilization bladders and inflation of one bladder of the plurality of inner stabilization bladders provides a three-point fixation to properly align the broken humerus bone to facilitate proper healing.

The present disclosure includes disclosure of an upper arm splint device, wherein inflation of two bladders of the plurality of inner stabilization bladders and inflation of one bladder of the plurality of outer stabilization bladders provides a three-point fixation to properly align the broken humerus bone to facilitate proper healing.

The present disclosure includes disclosure of an upper arm splint device, wherein the rigid outer member and the rigid inner member are configured to rotate radially at or about 90° around the shoulder/arm to accommodate stabilization for humeral breaks that occur in different axes/planes.

The present disclosure includes disclosure of an upper arm splint device, as shown and/or described herein.

The present disclosure includes disclosure of a method of stabilizing a broken humerus bone, comprising the steps of positioning an upper arm splint device about an upper arm of a user, the upper arm splint device, comprising a rigid outer member configured to fit upon an outside of an upper arm and shoulder, a rigid inner member configured to fit upon an inside of the upper arm, a plurality of outer stabilization bladders coupled to the rigid outer member and capable of independent inflation, a plurality of inner stabilization bladders coupled to the rigid inner member and capable of independent inflation, and a plurality of bands or straps connecting the rigid outer member to the rigid inner member, and inflating one or more bladders of the plurality of outer stabilization bladders and/or the plurality of inner stabilization bladders causes the broken humerus bone to properly align to facilitate proper healing.

The present disclosure includes disclosure of a method of stabilizing a broken humerus bone, as shown and/or described herein.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a splint device positioned about an upper arm, according to an exemplary embodiment of the present disclosure;

FIG. 2 shows a splint device positioned about an upper arm without any bladders inflated, according to an exemplary embodiment of the present disclosure;

FIG. 3 shows a splint device positioned about an upper arm with three bladders inflated, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

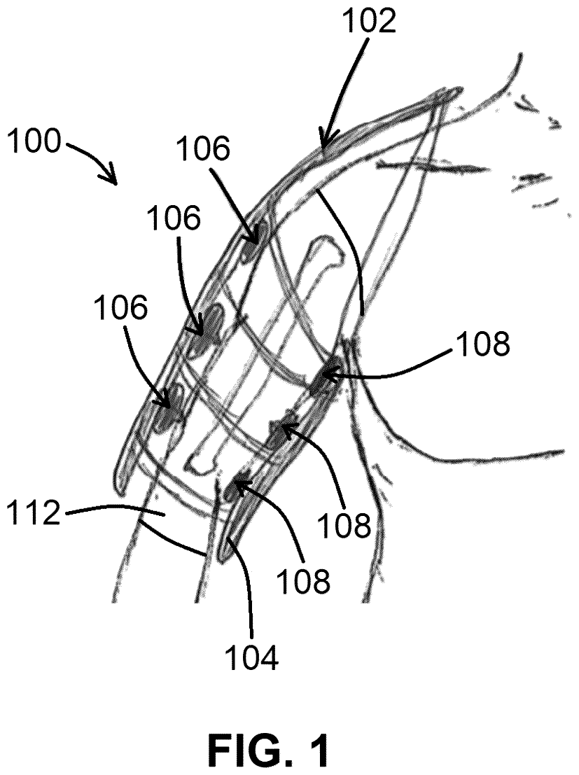

An exemplary upper arm humoral splint device 100 of the present disclosure is shown in FIG. 1. As shown therein, device 100 comprises a rigid outer member 102 and a rigid inner member 104. Said members 102, 104 (one or both) can comprise plastic or metal, or they (one or both) can comprise a rigid inflatable bladder that becomes rigid when inflated. As shown in FIG. 1, rigid outer member 102 is configured to extend between the shoulder and the distal end of the humerus bone of the user, while rigid inner member 104 is configured to extend between the armpit and the distal end of the humerus bone of the user.

A plurality of outer stabilization bladders 106 and a plurality of inner stabilization bladders 108 are configured to be positioned adjacent rigid outer member 102 and rigid inner member 104, respectively, so that said bladders 106, 108 can exert pressure against the arm of the user.

A plurality of bands or straps 110 extending between the rigid outer member 102 and the rigid inner member 104 can also be used so that the rigid outer member 102, the rigid inner member 104, and the bands or straps 110 completely encircle the upper arm. Bands or straps 110 can be permanently secured to one or both of the rigid outer member 102 and the rigid inner member 104, or they can be secured later, such as after when the rigid outer member 102 and the rigid inner member 104 are positioned relative to the upper arm.

In use, devices 100 of the present disclosure can be positioned so that they encircle the upper arm of the wearer. Once positioned, stabilization bladders 106, 108 can be inflated to cause pressure to be exerted against the upper arm and against the rigid outer member 102 and the rigid inner member 104, so to stabilize the upper arm, such as in the event of a break in the humerus bone. A sock, sleeve, or cloth 112, such as shown in FIG. 1, can be placed around the upper arm prior to positioning a device 100 of the present disclosure, so that the stabilization bladders 106, 108 contact said sock, sleeve, or cloth 112 when aligning a humerus bone, for example.

In at least one embodiment of the present disclosure, devices 100 comprise three outer stabilization bladders 106 and three inner stabilization bladders 108.

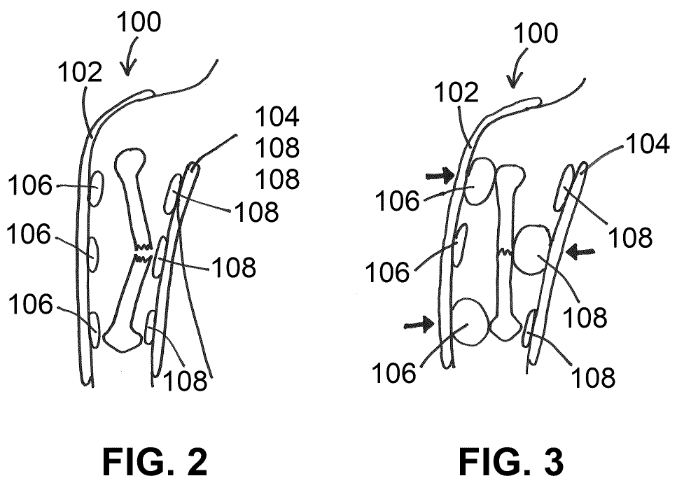

Devices 100 of the present disclosure are configured to provide “three point fixation” pressure against the upper arm when worn. For example, two outer bladders 106 and one inner bladder 108, or two inner bladders 108 and one outer bladder 106, can be inflated to provide three point fixation to stabilize the upper arm. In at least one example, a first and a third (the proximal and distal) outer bladders 106 and a second (the central) inner bladder 108 can be inflated to align a broken humerus bone, such as shown in FIG. 3 (with FIG. 2 showing bladders 106, 108 in an uninflated state). Alternatively, a second (the central) outer bladder 108 and a first and a third (the proximal and distal) inner bladders 108 can be inflated to align a broken humerus bone. Additional bladders 106, 108 can be inflated, such as a total of four, five, or six bladders 106, 108, as may be desired.

In at least some embodiments, inner bladders 108 are configured to rotate at or about 90° relative to the outer bladders 106. Such an embodiment would also allow for “three point fixation,” whereby the coronal plane is rotated 90° and then applied in the sagittal plane. In such embodiments, the outer and inner bladders 106, 108 can to rotate radially 90° around the shoulder/arm to accommodate stabilization for humeral breaks that occur in different axes/planes. In at least another embodiment, inner bladders 108 are configured to slide proximal or distal relative to the outer bladders 106 to accommodate location of the fracture and the length of the upper arm.

While various embodiments of devices for and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An upper arm splint device, comprising:
a rigid outer member configured to fit upon an outside of an upper arm and shoulder;
a rigid inner member configured to fit upon an inside of the upper arm;
a plurality of outer stabilization bladders coupled to the rigid outer member and capable of independent inflation;
a plurality of inner stabilization bladders coupled to the rigid inner member and capable of independent inflation; and
a plurality of bands or straps connecting the rigid outer member to the rigid inner member.

2. The splint device of claim 1, wherein the rigid outer member is capable of inflation, and wherein the rigid outer member becomes rigid upon inflation.

3. The splint device of claim 1, wherein the rigid inner member is capable of inflation, and wherein the rigid inner member becomes rigid upon inflation.

4. The splint device of claim 1, wherein the rigid outer member and/or the rigid inner member comprises plastic.

5. The splint device of claim 1, wherein the rigid outer member and/or the rigid inner member comprises metal.

6. The splint device of claim 1, wherein the plurality of bands or straps are coupled to the rigid outer member and are configured for subsequent connection to the rigid inner member.

7. The splint device of claim 1, wherein the plurality of bands or straps are coupled to the rigid inner member and are configured for subsequent connection to the rigid outer member.

8. The splint device of claim 1, configured to fit around an upper arm of a wearer having a broken humerus bone.

9. The splint device of claim 8, wherein inflation of one or more bladders of the plurality of outer stabilization bladders and/or the plurality of inner stabilization bladders causes the broken humerus bone to properly align to facilitate proper healing.

10. The splint device of claim 8, wherein inflation of two bladders of the plurality of outer stabilization bladders and inflation of one bladder of the plurality of inner stabilization bladders provides a three-point fixation to properly align the broken humerus bone to facilitate proper healing.

11. The splint device of claim 8, wherein inflation of two bladders of the plurality of inner stabilization bladders and inflation of one bladder of the plurality of outer stabilization bladders provides a three-point fixation to properly align the broken humerus bone to facilitate proper healing.

12. The splint device of claim 1, wherein the rigid outer member and the rigid inner member are configured to rotate radially at or about 90° around the shoulder/arm to accommodate stabilization for humeral breaks that occur in different axes/planes.

13. A method of stabilizing a broken humerus bone, comprising the steps of:
positioning an upper arm splint device about an upper arm of a user, the upper arm splint device, comprising:
a rigid outer member configured to fit upon an outside of an upper arm and shoulder,
a rigid inner member configured to fit upon an inside of the upper arm,
a plurality of outer stabilization bladders coupled to the rigid outer member and capable of independent inflation,
a plurality of inner stabilization bladders coupled to the rigid inner member and capable of independent inflation, and
a plurality of bands or straps connecting the rigid outer member to the rigid inner member; and
inflating one or more bladders of the plurality of outer stabilization bladders and/or the plurality of inner stabilization bladders causes the broken humerus bone to properly align to facilitate proper healing.
